(19) United States
(12) Patent Application Publication
(10) Pub. No.: US 2016/0360978 A1
(43) Pub. Date: Dec. 15, 2016

(54) Interferometric focusing beam optical cardiovascular sensor

(71) Applicant: Omnisensing Photonics LLC, Columbia, MD (US)

(72) Inventors: Wei Chen, Ellicott City, MD (US); Jin Zhang, Ellicott City, MD (US); Shan Zhong, Clarksville, MD (US); Zhonghua Zhu, Clarksville, MD (US)

(73) Assignee: Omnisensing Photonics LLC, Columbia, MD (US)

(21) Appl. No.: 15/146,354

(22) Filed: May 4, 2016

Related U.S. Application Data
(60) Provisional application No. 62/174,624, filed on Jun. 12, 2015.

(51) Int. Cl. A61B 5/026; A61B 5/00; A61B 5/021

(52) U.S. Cl. CPC A61B 5/0261; A61B 5/02125; A61B 5/7278; A61B 2562/0238; A61B 2562/04; A61B 2090/306

(57) ABSTRACT

This application discloses a potable, easy-to-use miniature cardiovascular sensor that is capable of monitoring heart rate, blood flow and blood pressure 24/7, using optical non-invasive method. It utilizes interferometric detection to improve signal to noise ratio. It also utilizes phase controlled focusing beam to reduce the optical power needed and therefore minimizing the power consumption, making it practical for continuous monitoring. The integrated optical chip assembly shrinks the total sensor size and makes it suitable for wearable devices, hence, this device will be portable and removable.

### FILED OF THE INVENTION

This application discloses a potable, easy-to-use miniature cardiovascular sensor that is capable of monitoring heart rate, blood flow and blood pressure 24/7, using optical non-invasive method. It utilizes interferometric detection to improve signal to noise ratio. It also utilizes phase controlled focusing beam to reduce the optical power needed and therefore minimizing the power consumption, making it practical for continuous monitoring. The integrated optical chip assembly shrinks the total sensor size and makes it suitable for wearable devices, hence, this device will be portable and removable.

### BACKGROUND OF THE INVENTION

The cardiovascular system is one of the most important vital systems in the human body. Monitoring its health will bring tremendous benefits for daily health care, cardiovascular function and risk assessment, disease prevention and health emergency alarming. The prevailing non-invasive method for cardiovascular system monitoring is Photoplethysmogram (PPG), which optically measures the pulsatile blood volumetric changes inside the blood vessel during each cardiac cycle when the heart pumps blood to the periphery. Even though this pressure pulse is somewhat dampened by the time it reaches the skin, it is enough to distend the arteries and arterioles in the subcutaneous tissue. The change in volume caused by the pressure pulse is detected by illuminating the skin with the light from a light-emitting diode (LED) and then measuring the amount of light reflected to a photo detector. Each cardiac cycle (heart beat) appears as a peak, and the repetition rate of the peak represents the heart rate.

The height of AC (Alternating Current) component of the photoplethysmogram is correlated with the pulse pressure. One may find it useful in calculating the systolic/diastolic blood pressure. However, since the AC component is also affected by the transmitter/receiver locations relative to the blood vessel, its accuracy and usefulness are greatly degraded. For reliably monitoring the heartbeat, the LED needs to illuminate a relatively large area of the skin. The photodiode also needs to collect lights reflected from a large area of the skin. Since only the light reflected from the blood vessel contains the information of the cardiac cycle, most optical power is wasted and therefore the power efficiency is relatively low.

### BRIEF DESCRIPTION OF THE INVENTION

In this invention, a near-infrared laser is used in the featured sensor for the cardiovascular system monitoring. This provides high directivity of the light to be able to focus the optical power on the blood vessel. It also enables the interferometric detection by combining the portion of the laser light with the reflected light. The phase controlled array waveguides can track and keep the laser beam focus on the blood vessel and therefore tolerate the relative movement between the sensor and the blood vessel, such that the optical power will be efficiently utilized, which subsequently reduces the amount of laser needed for illumination.

By placing two such sensors at a certain distance apart, we can measure the time interval between the passage of the arterial pulse wave at these two sensors sites during each cardiac cycle and calculate the Pulse Wave Velocity (PWV) from that. The PWV has been correlated with the relative blood pressure. Its measurement provides a reliable parameter for blood pressure monitoring.

### BRIEF DESCRIPTION OF THE DRAWINGS

The accompanying drawings, which are incorporated in and constitute a part of this specification, illustrate one or more implementations described herein and, together with the description, explain these implementations. In the drawings:

FIG. 1 is an exemplary diagram of a basic sensor configuration for methods described herein may be implemented;

FIG. 2 is an exemplary diagram of a duplex configuration of the sensor depicted in FIG. 1, for methods described herein may be implemented;

FIG. 3 is an exemplary film stack that the main body of the sensor may be built upon;

FIG. 4 illustrates an exemplary configuration for the sensor depicted in FIG. 1 to be used in a hand watch.

### DETAILED DESCRIPTION OF THE INVENTION

The following detailed description refers to the accompanying drawings. The same reference numbers in different drawings may identify the same or similar elements. Also, the following detailed description does not limit the invention.

### Overview

The penetration of light through human tissue has long been known and studied. Different imaging techniques, such as optical coherence tomography (OCT), laser Doppler flowmetry (LDF) and transmissive laser speckle imaging (TLSI), have been used in the medical practice. Penetration of living tissue depends on parameters like wavelength, intensity, polarization and coherence of the light source, tissue compression and those of the tissues themselves, like pigmentation, fibrotic structure, hydration and composition, in addition to more obvious factors such as hair and clothes.

The present invention is intended for a miniaturized sensor for continuous monitoring of the cardiovascular system.

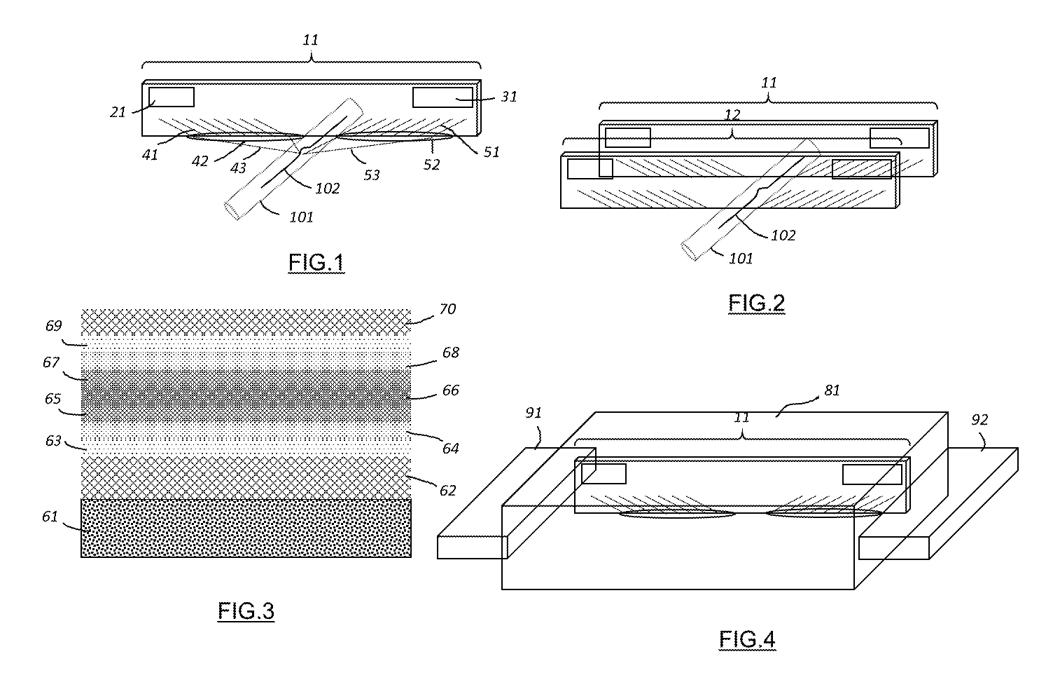

FIG. 1 is an exemplary diagram of a basic sensor configuration for methods described herein may be implemented. The near infrared light from the laser 21 is split into an array of waveguides 41, each having its individual phase controls. The outputs of the array waveguides are connected to micro lenses 42 fabricated on the sensor chip. Each light beam coming out of the lens has an elliptical propagation cone with a certain angle. And the phases from different beams are adjustable by the phase controls on the waveguides. One can control the phases so that only at certain far field location(s) a constructive interference takes place (a focus point). In the application the focus point is set to the predefined location relative to the artery/vein 101.

The reflected lights from the artery/vein are collected using the same focusing mechanism. The optical power collected by the collecting array waveguides 51 are recombined into a single waveguide and beat with a portion of the light that's tapped off from the laser 21. The collecting array waveguides 51 also have micro lenses 52. Since the coherent natural of the laser light, the tapped light and reflected light will interfere with each other and form an interferogram. The interferogram is recorded by the photo detectors 31, usually in a balanced configuration to eliminate the less useful DC components. As the optical path lengths for light reflected from the blood vessel change with the blood volume alteration inside it, the phase of reflected light also changes. The interferogram therefore contains the cardiac cycle information and can be used for the monitoring purpose.

A separate configuration for PWV measurement is shown in FIG. 2. By placing two such sensors 11, 12 a certain distance apart, we can measure blood flow rate and the Pulse Transit Time (PTT), i.e., the time it takes the pulsatile signal to propagate (travel) from one arterial site where the first sensor is located to the other site where the second sensor located during each cardiac cycle. The beat-to-beat PTT value can then be used to calculate PWV. As the speed at which arterial pulse wave travels is proportional to blood pressure, the PWV has been shown to correlate with blood pressure changes. For example, for a lateral displacement between sensors 11, 12 at 1 cm, and a typical PWV around 10 m/s, the PTT between two sensors is around 1 ms, which is readily detectable by the electronic circuitry. When blood pressure rises, it causes vascular tone to increase, hence the arterial wall becomes stiffer, while a stiffer vessel conducts pulse wave faster, this will result in a shortened PTT. Conversely, when blood pressure falls, PTT increases. Although absolute values of PWV or PTT cannot be well correlated with absolute values of blood pressure at a given point in time, PTT or PWV are capable of predicting changes in blood pressure over a short period of time, thus, serving as means of quantifying/detecting blood pressure changes associated with physical or mental activities or, more importantly, the blood pressure surges associated with cardiovascular diseases or health emergency condition.

### Sensor Fabrication Process

The main body of the sensor can be fabricated in a standard CMOS foundry.

On the main body chip, the waveguide core is defined by the photolithography process on the high refractive index (n>1.5) thin films. One candidate material is the Silicon Oxide Nitride (SiON), which is transparent in both the visible and most infrared wavelength ranges. The micro lenses are fabricated by multiple layers of differentiating refractive indices films. The phase controls were made from metal thin films deposited on top of the waveguide core. In order to have low power consumption, refractive indices of material from evanescent field of the optical mode will be tuned. A candidate for this material is Liquid Crystal (LC), which has demonstrated electric field dependent refractive index. The LC can be injected to a trench on the main chip defined by the photolithography and etch process.

The exemplary thin film stack is shown in FIG. 3. On a silicon substrate 61, a layer of silicon dioxide (SiO2) 62 is produced, usually by thermal oxidation. Additional layers of SiON 63-69 are then deposited on top of layer 62. Between the layer deposition, photolithography and etch process define the separate patterns within each layer.

### Application of the Sensor

In clinical practice, values of blood pressure are important markers of the cardiovascular status of patients, especially of those with hypertension. Although a cuff-based mercury sphygmomanometer continues to be the gold standard for diagnosing purposes, also, many cuff-based portable devices have been used for home blood pressure monitoring, the nature of this cuff-based technique does not allow for continuous monitoring of blood pressure as it causes discomfort by occluding and reopening blood vessels and disturbance to the patients, resulting in false readings of high blood pressure (white coat hypertension). However, blood pressure fluctuates throughout the day, even within hours or minutes under some extreme conditions. As cuff-based technique only gives single values at certain points of time, short-term or acute blood pressure fluctuations often cannot be detected by it at all. Most hypertension patient miss the early stage, because seldom anyone checks his blood pressure while laughing or crying, walking, exercising or during sex. Yet by measuring your blood pressure exactly at these times, you will know whether your blood pressure goes up or down within the safe range while occasional increase during physical or emotional stress often indicates an early tendency to hypertension. Most importantly, for hypertension patients, knowing and being alerted when their blood pressures are outside of the safe zone could reduce dramatically the risk of having stroke, heart attack or kidney failure, therefore greatly reducing hypertension-induced illness and death.

The sensor can be used on wearable devices, such as wristbands or watches. In the example configuration of FIG. 4, the sensor 11 is integrated in a hand watch by the watch case 81 and watch bands 91, 92. Other wearable device configurations are realizable too.

The featured device based on our present invention for blood pressure monitoring has the follows advantages:

Accurate BUT non-invasive, risk-free
Simple, easy to use (simple setup; self-measuring; home doable)
Convenient, portable and removable
Continuous (anti-vibration; even during sleep)
Cheaper than, BUT as reliable as current devices

### Health Risk

A near-IR laser is used in the sensor. Cataract and retinal burn could be caused by lasers in that wavelength range. However the power level of the laser is well below the Class-1 ANSI Laser Safety Standard (ANSI Z136.1) and should pose no known health risk on the human body.

### REFERENCES

[1] Heiko Gesche, Detlef Grosskurth, Gert Kuchler and Andreas Patzak, Continuous blood pressure measurement by using the pulse transit time: comparison to a cuff-based method, European journal of applied physiology, January 2012 112(1):309-15.
[2] Jacques Blacher, Roland Asmar, Saliha Djane, Gerard M. London and Michel E. Safar, Aortic Pulse Wave Velocity as a Marker of Cardiovascular Risk in Hypertensive Patients, Hypertension, 1999; 33: 1111-1117
[3] Incheol Jeong, Sukhwan Jun, Daeja Um, Joonghwan Oh and Hyungro Yooncorrespondinq, Non-Invasive Estimation of Systolic Blood Pressure and Diastolic Blood Pressure Using Photoplethysmograph Components, Yonsei medical journal, 2010

1. A cardiovascular system sensor comprising:
an integrated optical transmitter configured to have plurality of output optical waveguides that transmit laser lights to the blood vessels, each of plurality of the waveguide also include a phase control;
an integrated optical receiver configured to have plurality of input optical waveguides that receive laser lights reflected from the blood vessels, each of plurality of the waveguide also include a phase control.

2. The method of sensing operation of claim 1, where digital signal processing technique is used to decode the recorded signal of the cardiovascular system.

3. The transmitter of claim 1, where a portion of the laser light is tapped for the purpose of reference signal of the receiver of claim 1.

4. The receiver of claim 1, where reference and received laser lights interfere through an interferometric circuit and recorded by a photo detector or a pair of photo detectors in balanced configuration.

5. The transmitter of claim 1, where the plurality of output waveguides have integrated micro lenses.

6. The receiver of claim 1, where the plurality of input waveguides have integrated micro lenses.

7. The method of making the main body of such a sensor of claim 1, where multiple layers of high refractive index thin films were patterned to form a compact optical circuit.

8. The duplex configuration of claim 1, where two sensors are placed less than 5 cm apart.
